(19) United States
(12) Patent Application Publication
(10) Pub. No.: US 2025/0270643 A1
(43) Pub. Date: Aug. 28, 2025

(54) DETERMINING RISK OF FIBROMUSCULAR DYSPLASIA AND SYSTEMS AND METHODS OF USE THEREOF

(71) Applicant: The Regents of the University of Michigan, Ann Arbor, MI (US)

(72) Inventors: Santhi K. GANESH, Ann Arbor, MI (US); Xiang ZHOU, Ann Arbor, MI (US)

(21) Appl. No.: 18/858,246

(22) PCT Filed: Apr. 19, 2023
(86) PCT No.: PCT/US2023/019109

§ 371 Date: Oct. 18, 2024

Related U.S. Application Data
(60) Provisional application No. 63/332,526, filed on Apr. 19, 2022.

(51) Int. Cl. C12Q 1/6883; C12Q 1/6874

(52) U.S. Cl. CPC C12Q 1/6883; C12Q 1/6874; C12Q 2600/118; C12Q 2600/156

(57) ABSTRACT

Provided herein are systems and methods for determining a subject's risk of fibromuscular dysplasia (FMD) and/or abdominal aortic aneurysm (AAA), and methods of treatment and symptom management based thereon.

### CROSS-REFERENCE TO RELATED APPLICATIONS

This application claims the benefit of U.S. Provisional Patent Application No. 63/332,526, filed on Apr. 19, 2022, which is incorporated by reference herein.

### STATEMENT REGARDING FEDERAL FUNDING

This invention was made with government support under grant HL 139672 awarded by the National Institutes of Health. The government has certain rights in the invention.

### FIELD

Provided herein are systems and methods for determining a subject's risk of fibromuscular dysplasia (FMD) and/or abdominal aortic aneurysm (AAA), and methods of treatment and symptom management based thereon.

### BACKGROUND

Dysplasia-associated arterial disease (DAAD) encompasses a spectrum of non-atherosclerotic and non-inflammatory stenoses, dissections, and aneurysms affecting medium-sized muscular arteries, with excess burden on women, particularly those lacking otherwise common vascular disease risk factors. Multifocal fibromuscular dysplasia (FMD) is one form of DAAD, manifest by medial fibroplasia with excessive myofibroblasts amongst disorganized collagen and elastic fibrils (Refs. 1-5; incorporated by reference in their entireties), resulting in multiple arterial stenoses with intervening mural dilations leading to its “string of beads” appearance (Refs. 2-3 and 6-7; incorporated by reference in their entireties). FMD clinical manifestations often include refractory hypertension with renal artery involvement and strokes or transient ischemic attacks with cerebral, carotid, or vertebral artery involvement (Refs. 3, 8; incorporated by reference in their entireties). Patients with FMD may develop aneurysms or dissections in arteries exhibiting multifocal stenoses or they may occur in isolation (ref. 9, incorporated by reference in its entirety).

The earliest FMD pedigree studies supported autosomal dominant inheritance with incomplete penetrance (Refs. 1011; incorporated by reference in their entireties) but were limited by small sample size and lacked diagnostic imaging. More recently, a complex genetic basis of FMD has been associated with chromosome 6p24.1 PHACTR1, a locus similarly associated with spontaneous dissections of the carotid artery (CarAD) and coronary artery (SCAD)—both having been observed in patients with FMD (Refs. 12-14; incorporated by reference in their entireties). Data from the United States Registry for FMD and the National Institute on Aging suggest higher rates of vascular diseases among FMD relatives compared to the general population. An increased rate of sudden death was reported in the FMD registry, but correlates of this finding are unknown. Similarly, whether increased vascular diseases affect male relatives of FMD probands is unknown.

What is needed are symptoms and methods to determine the relative risks of arterial disease in family members of individuals with FMD.

### SUMMARY

Provided herein are systems and methods for determining a subject's risk of fibromuscular dysplasia (FMD) and/or abdominal aortic aneurysm (AAA), and methods of treatment and symptom management based thereon.

In some embodiments, provided herein are methods of assessing the risk of fibromuscular dysplasia (FMD) comprising: (a) testing a sample from the subject for biomarkers of FMD; and (b) assessing the subject's risk of FMD. In some embodiments, the biomarkers of FMD are selected from rs9349379, rs6580732, rs4719277, rs72802873, rs59610103, rs2616437, rs7895641, rs6711554, rs7072877, rs1341809, rs73437338, rs17489499, rs6442124, rs944797, rs73684699, rs11075223, rs7625090, rs7898456, rs2294476, rs2664354, rs73055680, rs11172113, rs6763419, rs342402, rs13196590, and rs12041871. In some embodiments, any combination of two or more (e.g., 2, 3, 4, 5, 6, 7, 9, 10, 11, 12, 13, 14, 15, 16, 17, 18, 19, 20, 21, 22, 23, 24, 25, or 26) of rs9349379, rs6580732, rs4719277, rs72802873, rs59610103, rs2616437, rs7895641, rs6711554, rs7072877, rs1341809, rs73437338, rs17489499, rs6442124, rs944797, rs73684699, rs11075223, rs7625090, rs7898456, rs2294476, rs2664354, rs73055680, rs11172113, rs6763419, rs342402, rs13196590, and rs12041871 are analyzed. In some embodiments, assessing the subject's risk of FMD comprises: (i) calculating a risk score based on the biomarkers for FMD; and (ii) comparing the risk score to a threshold to determine the subject's risk for FMD. In some embodiments, the presence of any biomarker is weighted according to its odds ratio in order to calculate the risk score. In some embodiments, the subject suffers from spontaneous coronary artery dissection (SCAD). In some embodiments, the subject is a human female. In some embodiments, the subject has a first or second degree relative that suffers from or has suffered from fibromuscular dysplasia (FMD) or AAA.

In some embodiments, provided herein are methods of assessing the risk of abdominal aortic aneurysm (AAA) comprising: (a) testing a sample from the subject for biomarkers of AAA; and (b) assessing the subject's risk of AAA. In some embodiments, the biomarkers of AAA are selected from rs11591147, rs646776, rs12730935, rs4916254, rs7255, rs1399623, rs7628052, rs3176336, rs7742931, rs118039278, rs10808546, rs4007642, rs7025486, rs1412445, rs501630, rs1892971, rs964184, rs4936098, rs1581613, rs11172113, rs7994761, rs1271512, rs55958997, rs4401144, rs73015016, rs429358, rs8124182, rs73149487, and rs2836411. In some embodiments, any combination of two or more (e.g., 2, 3, 4, 5, 6, 7, 9, 10, 11, 12, 13, 14, 15, 16, 17, 18, 19, 20, 21, 22, 23, 24, 25, 26, 27, 28, or 29) of rs11591147, rs646776, rs12730935, rs4916254, rs7255, rs1399623, rs7628052, rs3176336, rs7742931, rs118039278, rs10808546, rs4007642, rs7025486, rs1412445, rs501630, rs1892971, rs964184, rs4936098, rs1581613, rs11172113, rs7994761, rs1271512, rs55958997, rs4401144, rs73015016, rs429358, rs8124182, rs73149487, and rs2836411 are analyzed. In some embodiments, assessing the subject's risk of AAA comprises: (i) calculating a risk score based on the biomarkers for AAA; and (ii) comparing the risk score to a threshold to determine the subject's risk for AAA. In some embodiments, the presence of any biomarker is weighted according to its odds ratio in order to calculate the risk score. In some embodiments, the subject is a human male. In some embodiments, the subject as a first or second degree relative that suffers from or has suffered from fibromuscular dysplasia (FMD) or AAA.

In some embodiments, provided herein are methods of preventing fibromuscular dysplasia (FMD) and/or alleviating symptoms thereof comprising: (1) assessing the risk of FMD by the methods herein; and (2) administering a prophylactic and/or symptom management regime to the subject. In some embodiments, the prophylactic and/or symptom management regime comprises one or more of: (i) administering aspirin to the subject; (ii) cessation of tobacco use; (iii) balloon angioplasty; (iv) percutaneous transluminal angioplasty; (v) arteriogram; and (vi) administering antiplatelet therapy.

In some embodiments, provided herein are methods of preventing abdominal aortic aneurysm (AAA) and/or alleviating symptoms thereof comprising: (1) assessing the risk of AAA by the methods herein; and (2) administering a prophylactic and/or symptom management regime to reduce the subject. In some embodiments, the prophylactic and/or symptom management regime comprises one or more of: (i) administering statin to the subject; (ii) smoking cessation, (iii) ultrasound surveillance; (iv) avoiding activities that increase arterial strain or spikes in blood pressure; (v) monitoring blood pressure; (vi) controlling high blood pressure (e.g., administering a beta blocker; administering an angiotensin-converting enzyme inhibitor (ACEI); administering an angiotensin II receptor blocker (ARB), etc.), etc..

In some embodiments, provided herein are methods comprising (a) identifying a male subject as a first or second degree relative of a female individual that suffers from or has suffered from FMD; and (b) assessing the risk of AAA for the subject by the methods herein.

In some embodiments, provided herein are methods comprising: (a) identifying a female subject as a first or second degree relative of an individual that suffers from or has suffered from AAA; and (b) assessing the risk of FMD for the subject by the methods herein.

Embodiments of the present disclosure include a method of predicting FMD risk and/r AAA risk in a subject. In some embodiments, FMD risk is the likelihood that a subject will develop FMD. In some embodiments, FMD risk is the likelihood that a subject currently suffers from FMD. In some embodiments, AAA risk is the likelihood that a subject will develop AAA. In some embodiments, AAA risk is the likelihood that a subject currently suffers from AAA. In some embodiments, the subject being assessed for FMD risk is female. In some embodiments, the subject being assessed for AAA risk is male. In some embodiments, the subject has a first or second degree relative that suffers from or has suffered from or is at risk of FMD or AAA. In some embodiments, the subject exhibits symptoms of AAA or FMD.

In accordance with the embodiments herein, the methods include quantifying levels of at one or more biomarkers (e.g., single nucleotide polymorphisms (SNPs) from a sample from a subject; calculating a risk score based on the presence/absence of the one or more biomarkers; and determining subject's risk for AAA and/or FMD. In some embodiments, the subject is assigned a risk level, such as low risk, intermediate risk, or high risk of AAA and/or FMD based on the calculated risk score. In some embodiments, the biomarkers are weighted in the risk score calculation.

Embodiments of the present disclosure also include a biomarker panel for determining FMD risk in a subject. In accordance with these embodiments, the panel includes two or more (e.g., 2, 3, 4, 5, 6, 7, 9, 10, 11, 12, 13, 14, 15, 16, 17, 18, 19, 20, 21, 22, 23, 24, 25, or 26) of rs9349379, rs6580732, rs4719277, rs72802873, rs59610103, rs2616437, rs7895641, rs6711554, rs7072877, rs1341809, rs73437338, rs17489499, rs6442124, rs944797, rs73684699, rs11075223, rs7625090, rs7898456, rs2294476, rs2664354, rs73055680, rs11172113, rs6763419, rs342402, rs13196590, and rs12041871.

Embodiments of the present disclosure also include a biomarker panel for determining AAA risk in a subject. In accordance with these embodiments, the panel includes two or more (e.g., 2, 3, 4, 5, 6, 7, 9, 10, 11, 12, 13, 14, 15, 16, 17, 18, 19, 20, 21, 22, 23, 24, 25, 26, 27, 28, or 29) of rs11591147, rs646776, rs12730935, rs4916254, rs7255, rs1399623, rs7628052, rs3176336, rs7742931, rs118039278, rs10808546, rs4007642, rs7025486, rs1412445, rs501630, rs1892971, rs964184, rs4936098, rs1581613, rs11172113, rs7994761, rs1271512, rs55958997, rs4401144, rs73015016, rs429358, rs8124182, rs73149487, and rs2836411.

In some embodiments, the present disclosure provides a risk score, based on the presence/absence of one or more biomarkers (e.g., SNPs) to determine a subject's risk (e.g., low, intermediate, high, etc.) of AAA and/or FMD, thereby permitting selection of appropriate clinical management for the subject (e.g., therapies, surveillance, activity restrictions, etc.) . . .

### BRIEF DESCRIPTION OF THE DRAWINGS

FIG. 1. FMD and other DAAD on angiographic imaging and family pedigrees. (a) Catheter-directed angiography demonstrating multifocal FMD of the right renal artery. Arrows denote characteristic string of beads appearance. (b) Catheter-directed angiography depicting an isolated aneurysm involving a first order branch of the left renal artery. (c) Coronary angiography demonstrating SCAD. (d) Representative pedigrees of FMD families: dark shading identifies individuals affected with FMD including probands and relatives; vertical lines denote DAAD not formally diagnosed as FMD; dotted shading represents an individual with AAA; and unshaded relatives have no evidence of arterial dysplasia.

FIG. 2. Principal component analysis (PCA) plot of FMD cases and MGI controls Two-dimensional scatter plot of PC1 and PC2 from the PCA performed using the LASER/TRACE program to analyze FMD GWAS cases (Red dots, N=584), age, sex, PC-matched MGI controls (Black dots, N=7,193), and world-wide human genome diversity project (HGDP) samples as the reference, with different colors for different ethnicities as noted. In our FMD GWAS study, most FMD cases were European Ancestry (98%, N=572) and 2% of cases were African American (N=12).

FIG. 3. FMD GWAS Manhattan plot and QQ plot. Manhattan plot and QQ plot for the FMD GWAS result (N_{cases}=584, N_{controls}=7, 193) using SNPs with MAF≥1% and imputation Rsq≥0.8, based on saddlepoint approximation (SPA) corrected p-values of generalized mixed models in SAIGE, adjusted for PC1 to PC5. The sole variant meeting genome-wide Bonferroni-corrected significance threshold (P<5×10⁻⁸) is shown in blue. The λ_{GC} value is 0.95.

FIG. 4. Distributions of the PRS_{AAA} in FMD cases and controls. The distributions of the residual values of polygenic risk scores after adjusting for age, sex, PCs are shown in kernel density plots for comparison of cases versus control group. Residuals of PRS_{AAA} distribution (adjusted of age, sex and PC1-PC5) in FMD cases and MGI controls.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a biomarker” is a reference to one or more biomarkers and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including human and non-human animals (e.g., dogs, cats, cows, horses, sheep, poultry, fish, crustaceans, etc.). As used herein, the term “patient” typically refers to a subject that is being treated for a disease or condition.

As used herein, the term “first degree relative” refers to an individual that is a parent, child, or sibling of a subject.

As used herein, the term “second degree relative” refers to an individual that is a grandparent, aunt, uncle, first cousin, or half sibling of a subject.

As used herein, the term “preventing” refers to prophylactic steps taken to reduce the likelihood of a subject (e.g., an at-risk subject) from contracting or suffering from a particular disease, disorder, or condition. The likelihood of the disease, disorder, or condition occurring in the subject need not be reduced to zero for the preventing to occur; rather, if the steps reduce the risk of a disease, disorder or condition across a population, then the steps prevent the disease, disorder, or condition for an individual subject within the scope and meaning herein.

As used herein, the terms “treatment,” “treating,” and the like refer to obtaining a desired pharmacologic and/or physiologic effect against a particular disease, disorder, or condition. Preferably, the effect is therapeutic, i.e., the effect partially or completely cures the disease and/or adverse symptom attributable to the disease.

The terms “biological sample,” “sample,” and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), mucosal biopsy tissue and brushed cells, sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate (e.g., bronchoalveolar lavage), bronchial brushing, synovial fluid, joint aspirate, organ secretions, cells, a cellular extract, and cerebrospinal fluid. This also includes experimentally separated fractions of all of the foregoing. For example, a blood sample can be fractionated into serum, plasma, or into fractions containing particular types of blood cells, such as red blood cells or white blood cells (leukocytes). In some embodiments, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual. It will be appreciated that obtaining a biological sample from a subject may comprise extracting the biological sample directly from the subject or receiving the biological sample from a third party.

As used herein, the term “biomarker” refers to a measurable substance, the detection of which indicates a particular disease/condition or risk of acquiring/having a particular disease/condition. A “biomarker” may indicate a change in expression or state of the measurable substance that correlates with the prognosis of a disease. A “biomarker” may be a protein or peptide, a nucleic acid, or a small molecule. A “biomarker” may be measured in a bodily fluid such as plasma, and/or in a tissue (e.g., mammary tissue). In the context of the method described herein, a “biomarker” can be a single nucleotide polymorphism that is detected in a sample form a subject.

As used herein, the term “SNP” or “single nucleotide polymorphism” refers to a genetic variation between individuals; e.g., a single nitrogenous base position in the DNA of organisms that is variable. As used herein, “SNPs” is the plural of SNP. Of course, when one refers to DNA herein, such reference may include derivatives of the DNA such as amplicons, RNA transcripts thereof, etc. A “polymorphism” is a locus that is variable; that is, within a population, the nucleotide sequence at a polymorphism has more than one version or allele. One example of a polymorphism is a “single nucleotide polymorphism”, which is a polymorphism at a single nucleotide position in a genome (the nucleotide at the specified position varies between individuals or populations).

The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. An allele “positively” correlates with a trait when it is linked to it and when presence of the allele is an indicator that the trait or trait form will occur in an individual comprising the allele. An allele inversely correlates with a trait when it is linked to it and when presence of the allele is an indicator that a trait or trait form will not occur in an individual comprising the allele.

A marker polymorphism or allele is “correlated” or “associated” with a specified phenotype (e.g. FMD, etc.) when it can be statistically linked (positively or inversely) to the phenotype. That is, the specified polymorphism occurs more commonly in a case population (e.g., subjects suffering from FMD) than in a control population (e.g., subjects not suffering from FMD). This correlation is often inferred as being causal in nature, but it need not be—simple genetic linkage to (association with) a locus for a trait that underlies the phenotype is sufficient for correlation/association to occur.

The “polygenic risk score” is used to define an individuals' risk of developing a disease or condition, based on a multiple biomarkers, each of which might have modest individual effect sizes contribute to the disease or condition, but in aggregate have significant predicting value. In the present case, the polygenic risk score is used to predict the likelihood that a patient will develop or currently suffers from FMD. For example, the odds ratio (OR) from every variant is used to calculate the polygenic risk score. In some embodiments, the Odds Ratio for each variant present in a subject is multiplied by the number of reference alleles (0, 1 or 2) carried by the individual. In some embodiments, the resulting additive score is standardized to the same measure in population controls by the same measurement amongst population controls, resulting in the final polygenic risk score. Other methods of manipulating the odds ratios, presence/absence of the biomarkers, normalizing/standardizing the risk score, including controls, etc. are within the scope herein.

“Predetermined cutoff,” “cutoff,” “predetermined level,” and “reference level” as used herein refer to an assay cutoff value that is used to assess diagnostic, prognostic, or therapeutic efficacy results by comparing the assay results against the predetermined cutoff/level, where the predetermined cutoff/level already has been linked or associated with various clinical parameters. It is well-known that cutoff values may vary depending on the nature of the test, condition, etc. It further is well within the ordinary skill of one in the art to adapt the disclosure herein for tests, risk scores, and/or specific cutoff values based on the description provided by this disclosure. Whereas the precise value of the predetermined cutoff/level may vary between assays, the correlations as described herein should be generally applicable.

“Risk assessment,” “risk classification,” “risk identification,” or “risk stratification” of subjects (e.g., patients) as used herein refers to the evaluation of factors including biomarkers, to predict the risk of occurrence of future events (e.g., developing FMD, etc.) including disease onset or disease progression, or the likelihood that a current undiagnosed condition is present, so that treatment decisions regarding the subject may be made on a more informed basis.

As used herein, the terms “prognosis,” “prognosticate,” and related terms refer to the description of the likely outcome of a particular condition, such as the likelihood of FMD in a subject.

### DETAILED DESCRIPTION

Provided herein are systems and methods for determining a subject's risk of fibromuscular dysplasia (FMD) and/or abdominal aortic aneurysm (AAA), and methods of treatment and symptom management based thereon.

Experiments were conducted during development of embodiments herein to perform a study of 73 family pedigrees of probands with multifocal fibromuscular dysplasia (FMD) and 463 first degree relatives (total N study sample size N=536). An approximately 2× increased familial risk for an abdominal aortic aneurysm (AAA) was identified in fathers of probands. Fathers of probands having FMD and at least one arterial dissection had ˜3× increased risk of an AAA. No mothers of probands were found to have an AAA, and all relatives of FMD probands having an AAA were male. In the pedigree analysis, female relatives of FMD probands had a 2-3× increase in risk for FMD that was concentrated in younger generations. No male relatives had FMD. Using polygenic risk scores, a shared genetic basis for FMD and AAAs was verified. A similar shared basis for AAAs and dissection FMD-endophenotypes that are also part of the spectrum of dysplasia-associated arterial disease, spontaneous coronary artery dissection and carotid artery dissection, was also confirmed. 9.3% (43) of all first-degree relatives of probands were diagnosed with FMD, aneurysms, and dissections. Aneurysmal disease occurred in 60.5% of affected relatives and 5.6% of all relatives. Among 227 female first-degree relatives of probands, 4.8% (11) had FMD, representing a relative risk (RR)_{FMD} of 1.5 (95% CI:0.75-2.8, p-value=0.19) compared to the estimated population prevalence of 3.3%, though not of statistical significance. 11% (8 of 72) of FMD proband fathers had abdominal aortic aneurysms (AAAs) resulting in a RR_{AAA} of 2.3 (95% CI 1.12-4.6, P=0.014) compared to population estimates. The PRS_{FMD} was found to be associated with an AAA (OR=1.03[95% CI:1.01-1.05], P=2.6×10⁻³), and the PRS_{AAA} was found to be associated with FMD (OR=1.53[95% CI:1.2-1.9], P=9.0×10⁻⁵) as well. Experiments conducted during development of embodiments herein demonstrate the utility of screening for arterial vascular disease should be considered in relatives of individuals with FMD, specifically AAA screening among male relatives. FMD and AAAs are sex-dimorphic manifestations of a heritable arterial disease with a partially-shared complex genetic architecture. Excess risk of having an AAA according to a family history of FMD justifies screening of family members of individuals having FMD.

The disclosure provides a method comprising: (a) obtaining a biological sample from a subject (e.g., a subject suspected to be suffering from FMD, a subject with symptoms of FMD, a subject with a first or second degree relative that suffers from or has suffered from AAA or FMD); and (b) assaying the sample for one or more biomarkers described herein (e.g., one or more biomarkers of Table 6 (e.g., rs9349379, rs6580732, rs4719277, rs72802873, rs59610103, rs2616437, rs7895641, rs6711554, rs7072877, rs1341809, rs73437338, rs17489499, rs6442124, rs944797, rs73684699, rs11075223, rs7625090, rs7898456, rs2294476, rs2664354, rs73055680, rs11172113, rs6763419, rs342402, rs13196590, and rs12041871). As disclosed herein, the biological sample may be any biological material obtained or otherwise derived from an organism (e.g., a human). The biological sample may comprise, for example, saliva, blood, or a processed blood product. In some embodiments, obtaining a biological sample from a subject comprises extracting the biological sample directly from the subject or receiving the biological sample from a third party. In other embodiments, a biological sample may be extracted directly from a subject and sent to a third party for analysis.

The disclosure provides a method comprising: (a) obtaining a biological sample from a subject (e.g., a subject suspected to be suffering from AAA, a subject with symptoms of AAA, a subject with a first or second degree relative that suffers from or has suffered from AAA or FMD); and (b) assaying the sample for one or more biomarkers described herein (e.g., one or more biomarkers of Table 8 (e.g., rs11591147, rs646776, rs12730935, rs4916254, rs7255, rs1399623, rs7628052, rs3176336, rs7742931, rs118039278, rs10808546, rs4007642, rs7025486, rs1412445, rs501630, rs1892971, rs964184, rs4936098, rs1581613, rs11172113, rs7994761, rs1271512, rs55958997, rs4401144, rs73015016, rs429358, rs8124182, rs73149487, and rs2836411). As disclosed herein, the biological sample may be any biological material obtained or otherwise derived from an organism (e.g., a human). The biological sample may comprise, for example, saliva, blood, or a processed blood product. In some embodiments, obtaining a biological sample from a subject comprises extracting the biological sample directly from the subject or receiving the biological sample from a third party. In other embodiments, a biological sample may be extracted directly from a subject and sent to a third party for analysis.

In some embodiments, methods herein comprise calculating a risk score (e.g., risk of AAA (e.g., risk of having FMD, risk of developing FMD, etc.), risk of FMD (e.g., risk of having AAA, risk of developing AAA, etc.), etc.) based on the presence/absence of a combination of the biomarkers herein. In some embodiments, biomarkers contribution to the risk score is weighted by a factor related to the degree of correlation to a particular condition (e.g., FMD, AAA, etc.). In some embodiments, the biomarkers are weighted according to their effect estimate, odds ratio, or any other suitable measure of correlation. In some embodiments, a polygenic risk score is calculated.

Exemplary methods for detecting the presence or absence of a biomarker include, but are not limited to, polymerase chain reaction (PCR)-based technologies including, for example, reverse transcription PCR (RT-PCR) and quantitative or real-time RT-PCR (RT-qPCR). Other methods include microarray analysis, RNA sequencing (e.g., next-generation sequencing (NGS)), in situ hybridization, and Northern blot.

In some embodiments, nucleic acid (e.g., DNA or RNA) may be isolated, purified, and/or amplified from the biological sample prior to assaying the biological sample. Commercially available kits and systems for isolating and purifying nucleic acid (e.g., DNA or RNA) may be used in connection with the disclosure.

In some embodiments, primers, probes, or other reagents for detecting the biomarkers herein are provided. The polymorphisms, corresponding marker probes, amplicons or primers described herein can be embodied in any system herein, either in the form of physical nucleic acids, or in the form of system instructions that include sequence information for the nucleic acids. For example, the system can include primers or amplicons corresponding to (or that amplify a portion of) a gene or polymorphism described herein. As in the methods herein, the set of marker probes or primers optionally detects a plurality of polymorphisms. Thus, for example, the set of marker probes or primers detects at least one polymorphism in each of these polymorphisms or genes, or any other polymorphism, gene or locus defined herein. Any such probe or primer can include a nucleotide sequence of any such polymorphism or gene, or a complementary nucleic acid thereof, or a transcribed product thereof (e.g., a nRNA or mRNA form produced from a genomic sequence, e.g., by transcription or splicing).

In some embodiments, the risk score is compared to a threshold level and the subject is diagnosed as being at elevated risk or reduced risk of condition based thereon (e.g., elevated risk of having/developing FMD and/or AAA, reduced risk of having/developing FMD and/or AAA, etc.). The terms “threshold level” and “reference level” may be used interchangeably herein to refer to an assay value that is used to assess diagnostic, prognostic, or therapeutic efficacy and that has been linked or is associated herein with various clinical parameters. It is well-known that threshold levels may vary depending on the nature of the assay and that assays can be compared and standardized.

Embodiments involve detection and analysis of multiple genetic variants (e.g. SNPs) which are used to calculate a polygenic risk score suitable for identifying individuals at a greater or lesser risk of having or developing a condition (e.g., AAA, FMD, etc.). Detection methods for detecting relevant alleles include a variety of methods well known in the art, e.g., gene amplification technologies. For example, detection can include amplifying the polymorphism or a sequence associated therewith and detecting the resulting amplicon. This can include admixing an amplification primer or amplification primer pair with a nucleic acid template isolated from the organism or biological sample (e.g., comprising the SNP or other polymorphism), where the primer or primer pair is complementary or partially complementary to at least a portion of the target gene, or to a sequence proximal thereto. Amplification can be performed by DNA polymerization reaction (such as PCR, RT-PCR) comprising a polymerase and the template nucleic acid to generate the amplicon. The amplicon is detected by any available detection method, e.g., sequencing (e.g., next generation sequencing), hybridizing the amplicon to an array (or affixing the amplicon to an array and hybridizing probes to it), digesting the amplicon with a restriction enzyme (e.g., RFLP), real-time PCR analysis, single nucleotide extension, allele-specific hybridization, or the like. Genotyping can also be performed by other known techniques, such as using primer mass extension and MALDI-TOF mass spectrum (MS) analysis, such as the MassEXTEND methodology of Sequenom, San Diego, Calif. In certain embodiments, primers for amplification are located on a chip. Amplification can include performing a polymerase chain reaction (PCR), reverse transcriptase PCR (RT-PCR), or ligase chain reaction (LCR) using nucleic acid isolated from the organism or biological sample as a template in the PCR, RT-PCR, or LCR. In certain embodiments, the method further comprises cleaving the amplified nucleic acid. Other methods for detecting the biomarkers herein are understood in the field and applicable to embodiments herein.

In some embodiments, one or more additional steps are taken upon identifying a subject as having an elevated risk of developing or suffering from AAA and/or FMD. In some embodiments, methods further comprise a subsequent step of administering a treatment described herein. In some embodiments, methods further comprise cessation or avoidance of a treatment or activity that increases the risk of FMD or AAA and/or exacerbates symptoms or risks thereof. In some embodiments, methods further comprise additional monitoring, such as monitoring blood pressure, monitoring cardiac biomarkers, etc. In some embodiments, methods further comprise a subsequent step of screening said subject for comorbidities. In some embodiments, methods further comprise generating a report indicating the presence/absence of the biomarkers tested, a risk score generated, an elevated or reduced risk, and/or steps to be taken.

In some embodiments, a subject is administered an antiplatelet therapy based on the results of testing performed according to the methods described herein. Exemplary antiplatelet therapies include aspirin, clopidogrel, dipyridamole, etc.

In some embodiments, a subject is administered a beta blocker (beta-adrenergic blocking agent) based on the results of testing performed according to the methods described herein. Exemplary beta blockers include acebutolol, atenolol, betaxolol, betaxolol, bisoprolol fumarate, carteolol, carvedilol, esmolol, labetalol, metoprolol, nadolol, nebivolol, penbutolol, pindolol, propranolol, sotalol, and timolol.

In some embodiments, a subject is administered and angiotensin-converting enzyme (ACE) inhibitor and/or angiotensin II receptor blocker (ARB). Exemplary ACE inhibitors include benazepril (Lotensin), captopril., enalapril (Vasotec), fosinopril, lisinopril (Prinivil, Zestril), moexipril, perindopril, and quinapril (Accupril). Exemplary ARBs include azilsartan (Edarbi), candesartan (Atacand), eprosartan, irbesartan (Avapro), losartan (Cozaar), olmesartan (Benicar), telmisartan (Micardis), and valsartan (Diovan).

In some embodiments, method herein comprise counseling a subject at increased risk of FMD and/or AAA.

### EXPERIMENTAL

### Methods

Seventy-three unrelated consecutive adults with multifocal FMD evaluated at the University of Michigan Health System from 2012 to 2019 were enrolled in the University of Michigan (UM) Genetic Study of Arterial Dysplasia (HUM00044507). Clinical and family history data, including angiographic phenotypes were abstracted. Probands were defined as the first individual with multifocal FMD in a family to be enrolled in the study. Family histories to the level of first-degree relatives were collected by cardiovascular genetic counselors or physicians. DAAD diagnoses and evidence of potential DAAD were documented in probands and their family members.

### FMD Proband Clinical and Vascular Characteristics

Diagnosis required the identification of multifocal arterial beading in at least one vascular territory on angiographic imaging. These individuals were designated as “probands” for the purpose of our study. 73 probands enrolled through 2017 were included in the pedigree study. The electronic health record (EHR) and all available vascular imaging studies and study reports were reviewed for clinical characteristics and evidence of arterial dysplasia (aneurysm, dissection, or non-atherosclerotic focal stenosis). Health records and vascular imaging studies and study reports were requested from institutions outside the Michigan Medicine network where appropriate. Imaging studies sensitive to the detection of arterial dysplasia were designated as ‘vascular.’ These include: catheter-directed angiography (CA), contrast enhanced computed tomography (CECT), contrast enhanced magnetic resonance imaging (CEMRI), computed tomography angiography (CTA), and magnetic resonance angiography (MRA). All dysplastic arterial findings for the pedigree study were based on study evaluation by either an author specialized in vascular medicine or vascular surgery (SKG, JCS, DMC) or a board certified diagnostic or interventional radiologist by signed report. Proband clinical signs and symptoms were compiled from patient self-report via study questionnaires as well as manual audit of clinical notes where available.

### FMD Pedigree Construction and Analysis

Probands and family members were queried either directly or via a relative proxy for formal diagnoses of arterial dysplasia as FMD, aneurysm, dissection, including AAA, SCAD, and CeAD. Family members were also assessed for clinical signs of suspected undiagnosed DAAD which included, tabulated as “signs of FMD” in Table 3: cerebrovascular accident (CVA) at or before age 55, hypertension or antihypertensive medication requirement before age 30, renal artery stenosis (RAS) in the absence of cardiovascular disease, and/or sudden unexplained death without clear etiology. In addition to clinical data self-report or report via family member proxy, health records and vascular imaging studies and reports for first-degree family members were identified in the EHR or requested from outside institutions and reviewed as above.

Family member phenotypes were tallied and collapsed by relation to the proband for use in calculating relative risk scores by family relation. Familial relative risk was calculated by comparing the observed risk of first-degree relatives in our cohort to the total population estimate of a disease's prevalence. While all instances of DAAD and suspected signs of undiagnosed DAAD were reported, for the purposes of relative risk calculations our observations of FMD affected individuals were limited to first degree relatives with formally diagnosed FMD.

### Relative Risk and Confidence Intervals

The relative risk (RR) or the risk ratio was derived from RR=p1/p2-disease frequency in the test group/disease prevalence rate=x1/n1/x2/n2, where x1 is the number of cases in the test group, n1 is the total number of the test group, x2 is the number of cases in the whole population, and n2 is the total number of the whole population.

The confidence internal for the risk ratio was computed using the following equations.

$$SE \{ Ln ( \hat{RR} ) \} = \sqrt{\frac{( n 1 - x 1 ) / x 1}{n 1} + \frac{( n 2 - x 1 ) / x 2}{n 2}}$$

$$95 \% CI ( Ln ( \hat{RR} ) ) = Ln ( \hat{RR} ) \pm 1.96 \sqrt{\frac{( n 1 - x 1 ) / x 1}{n 1} + \frac{( n 2 - x 1 ) / x 2}{n 2}}$$

The confidence interval for Ln(RR) was converted to RR by finding the antilog of the result using exponential of the lower limit and the upper limit. A standard normal deviate (z-value) is calculated as {Ln( [symbol] )/SE{Ln( [symbol] )}, and the P-value is estimated from the area of the normal distribution that falls outside±z.

### Clinical Samples for Polygenic Risk Score (PRS) Analysis

### FMD Cases

Between 2010 and 2019, 584 individuals with multifocal FMD were enrolled in the UM Genetic Study of Arterial Dysplasia or the Cleveland Clinic FMD Biorepository. This included 73 probands whose pedigrees were studied. Informed consents and study activities were approved by the enrolling institution's IRB.

### MGI Controls for FMD GWAS

The Michigan Genomics Initiative (MGI) included participants who consented for genetic analysis of their blood and access of medical information from their electronic health record (EHR) (Ref. 54; incorporated by reference in its entirety). The current study involved 13,756 individuals from MGI, excluding those having ICD codes of vascular diseases and connective tissue disorders (Ref. 14; incorporated by reference in its entirety).

Each FMD research participant included in the University of Michigan Genetic Study of Arterial Dysplasia or the Cleveland Clinic FMD Biorepository contributed either a blood or saliva sample via standard K+EDTA blood collection tubes or commercial saliva collection kits (Oragene, DNAGenotek). DNA was isolated according to commercial kit protocols (Nucleospin Tissue (TakaraBio)), extracted according to the prepIT-L2P extraction kit (DNAGenotek) and quantified using the Quant-iT PicoGreen dsDNA kit (ThermoFisher).

### Million Veteran Program AAA Analysis

The Veterans Affairs Million Veteran Program (MVP) (Ref. 55; incorporated by reference in its entirety) included more than 825,000 active users of the Veteran Health Administration (VA) since 2011 from more than 60 VA Medical Centers nationwide. Participants consented to provide blood for genomic analysis and access to their full EHR data. Imputed genetic information was available from 314,434 participants assigned to white-European ancestry using the HARE algorithm (Refs. 56-57; incorporated by reference in their entireties). International Classification of Diseases (ICD9/10) diagnostic and Current Procedural Terminology (CPT) codes allowed identification of patients with AAA (Refs. 29, 58; incorporated by reference in their entireties). The algorithm identified 9,693 unrelated subjects with AAA and 294,049 unrelated controls (3,257 subjects were excluded with ambiguous AAA status). Genotyping, genome-wide association analysis of AAA, and association results have been previously reported (Refs. 29, 58; incorporated by reference in their entireties).

### UK Biobank AAA Analysis

The UK Biobank (UKB) cohort included 486,906 samples with complete genotypic and phenotypic data without missing covariates information. Ancestry of these samples estimated according to genotype-derived PC's was that were 97.6% European ancestry, 0.6% Asian ancestry, and 1.8% African ancestry. AAA phenotype was defined as has been performed previously using ICD codes, which identified 998 individuals with AAA (876 males, and 122 females) and 485,908 controls (221,966 males, 263,942 females). Among the 998 AAA cases, 99.3% was European ancestry, 0.1% Asian ancestry, and 0.6% African ancestry.

### Genotyping and SNV Association Analysis of FMD

Genotyping of all FMD and MGI samples were conducted by the UM DNA Sequencing Core. Case-control match was accomplished for age, sex and ancestry. The FMD GWAS used a case control ratio up to 1:14, including 584 FMD cases and 7,193 matched controls. GWAS analysis included the first five principal components as covariates and was implemented in SAIGE (Ref. 59; incorporated by reference in its entirety) to minimize type I error rates due to case-control imbalance and sample relatedness.

FMD cases and MGI controls were both genotyped with the same version (v1.1) of the Illumina BeadArray, using the Illumina Infinium HTS Assay Protocol, a semi-custom Infinium CoreExome-24v1.1 BeadArray with 607,778 SNP markers (UM_HUNT_Biobank_v1-1_20006200_A), and the Illumina GenomeStudio v2011.1., utilizing a GWAS+exome chip platform which includes standard genome-wide tagging SNPs (N˜240,000), exoNic variants (n˜280,000) and custom content from previously published GWASs, additional exonic variants selected from sequencing studies, ancestry informative variants and Neanderthal variants. Data Analysis Software package with Genotyping Module v1.9.4 and Illumina GenomeStudio (version 2.0) were used to cluster and call genotypes. Sample filtering was performed to exclude samples with call rate <98%, estimated contamination >2.5% (BAF regress), chromosomal missingness greater than 5 times other chromosomes, and sex mismatch between genotype-inferred sex and reported gender. Variant filtering was performed to exclude probes that could not be perfectly mapped to the human genome assembly (Genome Reference Consortium Human genome build 37 and revised Cambridge Reference Sequence of the human mitochondrial DNA; BLAT); Hardy Weinberg equilibrium deviations in European ancestry samples (P<0.00001); variant call rate <98%.

Following the QC and merging of the data sets as described in the text, 351,487 polymorphic variants remained (chr1-22). The total genotyping rate was 0.99. Two samples were removed due to gender mismatch and eight were removed because they were found to be duplicated samples. Relatives were retained for analysis as the generalized mixed model accounts for sample relatedness. QC steps were implemented in PLINK. We then imputed autosomal chromosome genotypes of the Haplotype Reference Consortium (HRC) using the Michigan Imputation Server on the 13,756 MGI and 584 FMD samples. The parameters for imputation included: 1) Minimac4 method; 2) HRC r1.1 2016 reference panel; 3) Eagle v2.3 as phase output; 4) EUR as quality control population. Poorly imputed variants (R2<0.8) and rare variants (MAF<1%) were filtered. SNPs with potential frequency mismatches were also excluded by comparing with reference panel (markers with Chi-squared greater than 300). 6,604,766 imputed variants (chromosomes 1-22) were retained after the filter. The correlation r2 between the reference allele frequency of the samples and the HRC reference panel was 0.999. GWAS analysis was implemented in SAIGE which uses mixed models to account for relatedness and introduces a scalable and accurate generalized mixed model association test that utilizes the saddle-point approximation to calibrate the distribution of score test statistics (minimizing type I error rates due to case-control imbalance and sample relatedness).

### Ancestry Estimation by Principal Components Analysis

TRACE in LASER (Locating Ancestry from SEquence Reads) software v3.0.0 was used to compute 5 principal components based on the genotype data to map the individual's genetic ancestry using world-wide HGDP samples as reference (Ref. 60; incorporated by reference in its entirety). Age, sex, and ancestry matched controls were selected from MGI based upon the top 3 PCs given by TRACE program in LASER server (Refs. 61-63; incorporated by reference in their entireties). The FMD cohort consisted of 98.2% European ancestry and 1.8% African American individuals.

PRS Construction and Statistical Analysis of PRS Associations with FMD and AAA

The FMD GWAS results (MAF≥0.01) were LD-pruned for SNPs with r2≤0.3 within +/−500 Kb of the index SNP in each region, to define independent loci. A PRS_{FMD} was constructed with independent SNPs meeting a false discovery rate q value <0.1 (26 loci), from 408,241 LD-pruned genome wide loci. The PRS_{AAA} had been previously defined to include 29 AAA SNPs identified by analyses of MVP (Ref. 29; incorporated by reference in its entirety). The PRS_{CarAD} was defined based upon published loci associated with CarAD (Ref. 12; incorporated by reference in its entirety), and similarly the PRS_{SCAD} has been previously published (Ref. 14; incorporated by reference in its entirety).

The 29-SNP PRS_{AAA} was tested for association using the GTX package in R (Refs. 64-66; incorporated by reference in their entireties), which relied on GWAS summary statistics of the primary SAIGE-based FMD GWAS. For the analysis of the PRS_{FMD}, PRS_{CeAD}, and PRS_{SCAD} associations with AAA in MVP, logistic regression was utilized to estimate the association between the polygenic risk score and the disease, adjusting for age, sex, and the first 5 principal components as covariates. A similar PRS_{FMD} association analysis of AAA was also conducted in UKB. Weighted polygenic scores were calculated as a continuous variable using the equation: Σ(β1*SNP1)+ . . . + (βn*SNPn), where βx denotes the beta coefficient of the effect allele, which are single SNP regression coefficients estimated for the effect allele in the discovery GWAS dataset. SNPx denotes the number of effect alleles (0, 1, or 2).

The imputed data sets (incorporating directly genotyped and imputed data) were utilized to conduct GWAS and PRS analyses. The summary statistics of FMD were obtained from a generalized mixed model that utilizes the saddle-point approximation to reduce the type I error rates due to case-control imbalance, adjusted for PC1 to PC5. The summary statistics of AAA were generated from logistic regression, adjusted for age of last visit, sex (if applicable), and PC1 to PC10. As a sensitivity analysis. logistic regression using individual genotyping data was also conducted for PRS-AAA in the FMD cohort, adjusted for age, sex, and the first five principal components of genetic ancestry. The PRS_{AAA} for FMD using logistic regression to analyze individual genotype data was based upon a lower (1:6) case:control ratio as compared to the FMD GWAS conducted with SAIGE which allowed for a higher (1:14) FMD case:control ratio and the inclusion of related samples but had the drawback of yielding potentially down-weighted effect estimates due to corrections implemented in the SAIGE algorithm that are effective at controlling inflation. PRS analyses using either summary statistics by GTX or standard logistic regression yielded similar results.

### Results

### FMD Clinical Characteristics and Familial Relative Risk (RR)

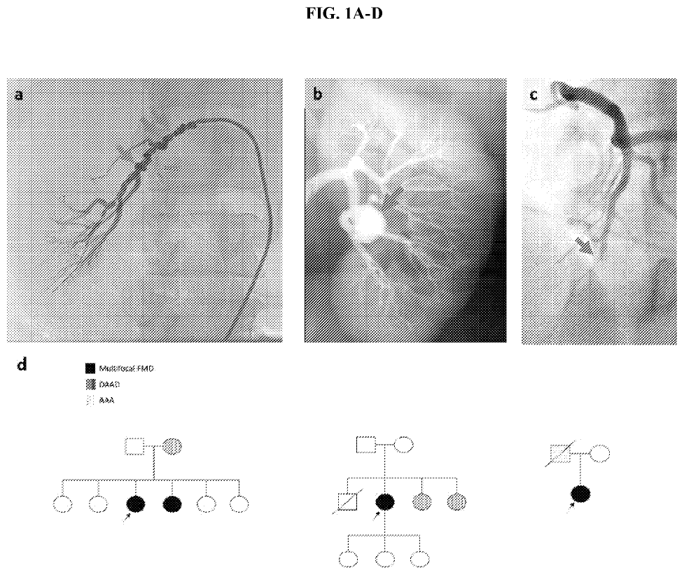

Arterial phenotypes (FIG. 1), clinical phenotypes, and family pedigrees were constructed for the study's 73 FMD probands (Table 1). Family member data were obtained by self-reporting with imaging confirmation when possible, including all relatives with a formal diagnosis of FMD (online supplement). FMD Probands were all female with an average age at diagnosis of 49±10 years, and 93.2% were of European ancestry. Hypertension affected 73% of the probands and 26% were former or current smokers (Table 1).

TABLE 1
Clinical characteristics of probands with multifocal FMD
from the pedigree study. Values are presented as N (%)
or mean ± standard deviation [range]. Denominator
of reported percentages is given if different than total
cohort size of N = 73 to account for incomplete data .

| Variable | All, N (%) |
|---|---|
| N | 73 |
| Female | 73 (100) |
| White | 68 (93.2) |
| Body mass index | 25.4 ± 5.5 [16.7, 46.3] |
| Age at clinical presentation | 44.4 ± 13.7 [12, 75] |
| Age at FMD diagnosis | 49.0 ± 10.4 [13, 75] |
| Comorbidities | |
| Hypertension | 53 (72.6) |
| Hyperlipidemia | 28 (38.4) |
| Smoking history | 19 (26.0) |
| Diabetes | 2 (2.7) |
| Coronary artery disease/Myocardial infarction | 7 (9.6) |
| History of stroke | 5 (6.9) |
| History of transient ischemic attack | 13 (17.8) |
| Clinical Signs & Symptoms | |
| Headache | 59 (83.1) |
| Dizziness | 40 (56.3) |
| Tinnitus (pulsatile or nonpulsatile) | 38 (53.5) |
| Cervical bruit | 23 (32.4) |
| Neck pain | 35 (49.3) |
| Abdominal bruit | 12 (16.9) |
| Post prandial abdominal pain | 16 (22.5) |
| Flank pain | 20 (28.2) |
| Abdominal pain | 21 (29.6) |
| Renal infarction | 7 (9.9) |
| Claudication | 15 (21.1) |
| Chest pain | 16 (22.5) |
| Horners syndrome | 4 (5.6) |
| Amaurosis fugax | 9 (12.7) |
| History of myocardial infarction | 3 (4.2) |
| Mesenteric ischemia | 3 (4.2) |

A total of 463 first-degree relatives were evaluated for DAAD phenotypes, inclusive of multifocal FMD, aneurysms, dissections, and suspected signs of undiagnosed DAAD (Table 2, Table 3). Only one proband's parental history was unknown. DAAD was definitively diagnosed in 9.3% (46) of relatives, being aneurysmal disease in 60.5% of relatives with a DAAD and 5.6% of all relatives.

TABLE 2
Phenotypes of interest in first degree relatives by family relation
and sex. Arterial aneurysms and arterial dissections represent
individuals with these findings consistent with DAAD, but not
formal diagnoses of FMD. Phenotypes are mutually exclusive (for
example, if a family member was diagnosed with FMD and was also
known to have an arterial aneurysm, that individual would be
counted as ‘FMD’ but not ‘Arterial aneurysm’. Suspected
DAAD are clinical signs that may represent undiagnosed DAAD which
included: stroke or transient ischemic attack at or before age
55, hypertension or antihypertensive medication requirement before
age 30, renal artery stenosis in the absence of cardiovascular
disease, and/or sudden unexplained death without clear etiology.
All relatives are the sum of all first-degree relatives of probands,
regardless of clinical status.

| | | Arterial | Arterial | Suspected | |
|---|---|---|---|---|---|
| | FMD | aneurysms | dissections | DAAD | All relatives |
| Parent | | | | | |
| F | 2 | 3 | 1 | 6 | 72 |
| M | 0 | 12 | 1 | 6 | 72 |
| Total | 2 | 15 | 2 | 12 | 144 |
| Sibling | | | | | |
| F | 4 | 4 | 1 | 5 | 91 |
| M | 0 | 5 | 1 | 7 | 94 |
| Total | 4 | 9 | 2 | 12 | 185 |
| Offspring | | | | | |
| F | 5 | 1 | 2 | 0 | 64 |
| M | 0 | 1 | 0 | 6 | 70 |
| Total | 5 | 2 | 2 | 6 | 134 |
| All First-Degree | | | | | |
| Relatives | | | | | |
| F | 11 | 8 | 4 | 11 | 227 |
| M | 0 | 18 | 2 | 19 | 236 |
| Total | 11 | 26 | 6 | 30 | 463 |

TABLE 3
Arterial beds affected by aneurysm in first degree relatives
by family relation and sex. Individuals included in
this table did not carry a diagnosis of FMD.

| | Abdominal | Thoracic | Cerebral | Other or not | |
|---|---|---|---|---|---|
| | aorta | Aorta | aneurysm | specified | Total |
| Parent | | | | | |
| F | 0 | 0 | 2 | 1 | 3 |
| M | 8 | 0 | 2 | 2 | 12 |
| Total | 8 | 0 | 4 | 3 | 15 |
| Sibling | | | | | |
| F | 0 | 0 | 2 | 2 | 4 |
| M | 1 | 0 | 2 | 2 | 5 |
| Total | 1 | 0 | 4 | 4 | 9 |
| Offspring | | | | | |
| F | 0 | 1 | 0 | 0 | 1 |
| M | 0 | 0 | 1 | 0 | 1 |
| Total | 0 | 1 | 1 | 0 | 2 |
| All First-Degree | | | | | |
| Relatives | | | | | |
| F | 0 | 1 | 4 | 3 | 8 |
| M | 9 | 0 | 5 | 4 | 18 |
| Total | 9 | 1 | 9 | 7 | 26 |

Eleven first-degree relatives were diagnosed with FMD; all affecting female members, including 2 mothers, 4 sisters, and 5 daughters. The RR_{FMD} among all female first-degree relatives was 1.45 (95% CI:0.75-2.82) (Table 4). Analyses of the RR_{FMD} focused on siblings and children in whom diagnostic imaging had been undertaken according to current clinical practice. Among female siblings and offspring, the RR_{FMD} was 1.74 (95% CI:0.85-3.56). Among female siblings and offspring of a subset of probands having a more severe FMD phenotype (aneurysms or dissections) the RR_{FMD} was 2.5 (95% CI:1.2-5.4) (Table 5).

TABLE 4
Familial relative risk for FMD and AAA, as well as absolute risk for DAAD (which includes FMD, AAA,
non-abdominal aortic aneurysm, and arterial dissection). Relative risk (RR) and 95% confidence intervals
(CI) were computed based upon the distribution of the individuals in each category with the listed findings
(n) as compared to the total number of individuals per category (N). Population prevalence (P) of 3.34%
for FMD16 and 4.9% for AAA^{17, 51-53} was used to calculate relative risk. The absolute risk is represented
by the probability that a relative is affected (p = n/N).

| | | | | | | | | | Family Members | | |
|---|---|---|---|---|---|---|---|---|---|---|---|
| | | | | | | | | | with diagnosed | | |
| | Family members with FMD | | | | Family members with AAA | | | | DAAD + AAA | | |
| | | | p = | | | | p = | | | | p = |
| | n | N | n/N | RR [95% CI] | n | N | n/N | RR [95% CI] | n | N | n/N |
| Female Parents | 2 | 72 | 2.80% | 0.83 [.20-3.40] | 0 | 72 | — | | 6 | 72 | 8.30% |
| Male Parents | 0 | 72 | — | | 8 | 72 | 11.10% | 2.27 [1.12-4.60] | 13 | 72 | 18.10% |
| Female Siblings | 4 | 91 | 4.40% | 1.32 [.48-3.63] | 0 | 91 | — | | 9 | 91 | 9.90% |
| Male Siblings | 0 | 94 | — | | 0 | 94 | — | | 6 | 94 | 6.40% |
| Female Offspring | 5 | 64 | 7.80% | 2.34 [.95-5.78] | 0 | 64 | — | | 8 | 64 | 12.50% |
| Male Offspring | 0 | 70 | — | | 0 | 70 | — | | 1 | 70 | 1.40% |
| All Relatives | 11 | 463 | 2.40% | 0.71 [.36-1.39] | 8 | 463 | 1.70% | 0.35 [.17-.74] | 43 | 463 | 9.30% |
| Male Relatives | 0 | 236 | — | | 8 | 236 | 3.40% | 0.69 [.33-1.44] | 20 | 236 | 8.50% |
| Female Relatives | 11 | 227 | 4.80% | 1.45 [.75-2.82] | 0 | 227 | — | | 23 | 227 | 10.10% |
| Male Siblings + | 0 | 164 | — | | 0 | 164 | — | | 7 | 164 | 4.30% |
| Male Offspring | | | | | | | | | | | |
| Female Siblings + | 9 | 155 | 5.80% | 1.74 [.85-3.56] | 0 | 155 | — | | 17 | 155 | 11.00% |
| Female Offspring | | | | | | | | | | | |

TABLE 5
Familial relative risk for FMD and AAA among family members of probands with FMD and
at least one aneurysm or dissection. Relative risk (RR) and 95% confidence intervals
(CI) were computed based upon the distribution of the individuals in each category with
the listed findings (n) as compared to the total number of individuals per category
(N). Population prevalence (P) of 3.34% for FMD and 4.9% for AAA was used to calculate
relative risk, as described in the text. Absolute Risk (AR) difference compared to the
population prevalence is shown, with negative values indicative of elevated risk.

| | Relatives with FMD | | | | Family member with AAA | | | |
|---|---|---|---|---|---|---|---|---|
| — | n | N | p = n/N | RR [95% CI] | n | N | p = n/N | RR [95% CI] |
| Female Parents | 1 | 45 | 2.20% | 0.7 [0.1-4.8] | 0 | 45 | — | |
| Male Parents | 0 | 45 | — | | 5 | 45 | 11.10% | 2.3 [0.9-5.4] |
| Female Siblings | 4 | 57 | 7.00% | 2.1 [0.8-5.7] | 0 | 57 | — | |
| Male Siblings | 0 | 61 | — | | 0 | 61 | — | |
| Female Offspring | 4 | 37 | 10.80% | 3.2 [1.2-8.7] | 0 | 37 | — | |
| Male Offspring | 0 | 41 | — | | 0 | 41 | — | |
| Relatives | 9 | 286 | 3.10% | 0.9 [0.5-1.9] | 5 | 286 | 1.70% | 0.4 [0.1-0.9] |
| Male Relatives | 0 | 147 | — | | 5 | 147 | 3.40% | 0.7 [0.3-1.7] |
| Female Relatives | 9 | 139 | 6.50% | 1.9 [0.9-4.0] | 0 | 139 | — | |
| Male Siblings + | 0 | 102 | — | | 0 | 102 | — | |
| Male Offspring | | | | | | | | |
| Female Siblings + | 8 | 94 | 8.50% | 2.5 [1.2-5.41] | 0 | 94 | — | |
| Female Offspring | | | | | | | | |

Risks may be underestimated for the older relatives given that it is less likely that angiographic imaging was performed or available in these cases. In that regard it was found 2 of 72 mothers (2.8%) diagnosed with FMD (RR_{FMD}=0.83) (95% CI:0.20-3.40) and another 6 of 72 mothers with suspected DAAD but without a formal diagnosis. Notably, male relatives did have signs of a suspected DAAD diagnosis, with 19 of 236 (8.1%) of all male relatives being affected. Among female relatives, 11 of 227 (4.8%) were diagnosed with FMD and an additional 11 of 227 (4.8%) had suspected DAAD (Table 2). The relative risks for suspected DAAD in family members was not calculated, given the uncertainty of the population prevalence of signs consistent with suspected DAAD, and whether DAAD actually represented undiagnosed FMD.

Among all first-degree relatives of probands, 5.6% (26) had an arterial (including aortic) aneurysm without a diagnosis of FMD (Table 3), supporting the characterization of DAAD as a set of related but variable arterial phenotypes having a shared underlying predisposition. The presence of aneurysmal disease, in any arterial bed, was more common (N=18/26, 69%) in the proband's male family members, and among proband fathers, 8 AAAs were identified that corresponded to a relative risk of having an AAA (RR_{AAA}) of 2.3 (95% CI:1.1-4.6) (Table 4). No mother of a proband had an AAA (Table 3). Among a subset of families in which the proband had at least one aneurysm or dissection, the RR_{AAA} was unchanged at 2.3 (95% CI:0.95-5.4) (Table 5).

### Genetic Risk Scores to Interrogate the Relationship Between FMD and AAAs

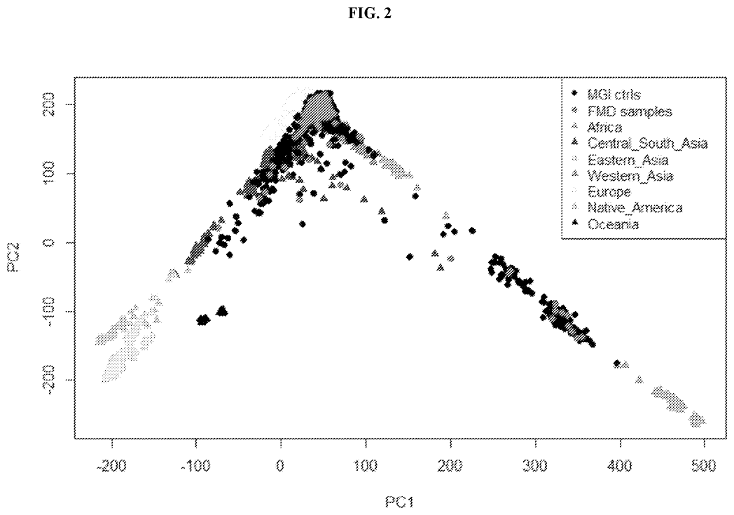

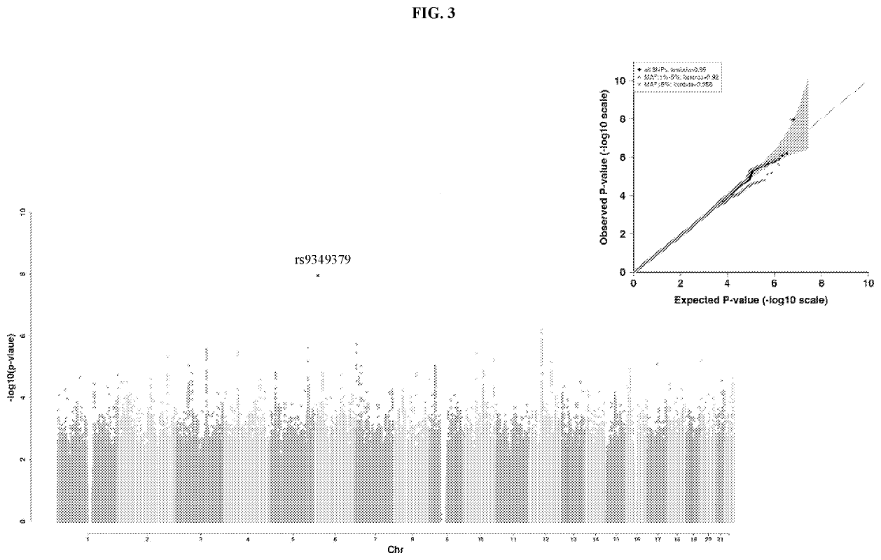

The complex genetic architecture of FMD was first assessed with a GWAS based upon 584 FMD cases and 13,756 MGI controls subjects. The average age of the FMD cases was 53±12 years, 96.2% were female, 97.2% were self-reported European Ancestry, and 2.1% were African American (FIG. 2). 7,193 MGI controls were selected to match for age, sex, and ancestry; average age was 52±16 years, and 95.8% were female. The analysis was combined across ethnicity, and using principal components (PC1-PC3) estimated from LASER/TRACE program to map against HGDP samples, the samples used in our analyses were 98.2% European ancestry and 1.8% African American. Post-imputation filtering for SNPs with r²≥0.8 and MAF≥0.01 yielded 6,604,767 SNPs for association testing (FIG. 3). There was no evidence of genomic inflation of association statistics which would lead to false positives (λ_{GC}=0.95). The previously identified FMD-associated chromosome 6 PHACTR1 locus rs9349379(A) was the single locus meeting genome-wide significance (odds ratio, OR=1.4[95% CI:1.3-1.6], P=1.10×10⁻⁸). The PRS_{FMD} constructed from the FMD GWAS, including independent SNPs with FDR q-value<0.1, was comprised of 26 SNPs (Table 2).

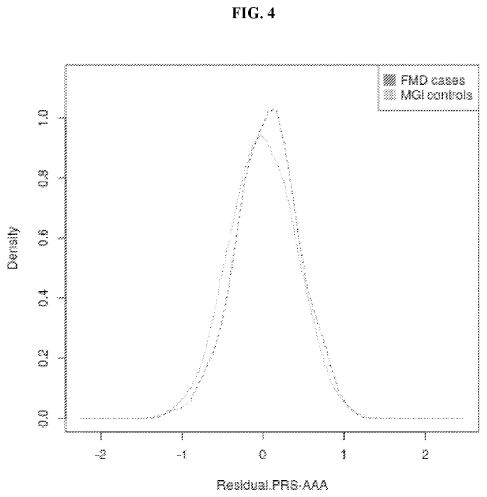

A PRS_{AAA} previously defined in the individuals with white-European ancestry from MVP cohort including 29 AAA-associated independent SNPs was associated with higher risk of FMD using the R package Genetics Toolbox (GTX), which uses summary statistics obtained from the primary FMD GWAS analyzed with SAIGE (OR=1.5[95% CI:1.2-1.9], P=9.0×10⁻⁵) (FIG. 4, Table 7, Table 8). The result was similar in a sensitivity analysis using logistic regression to analyze individual genotype data (OR=1.5[95% CI:1.2-1.9], P=3.0×10⁻⁴). Similarly, the PRS_{FMD} was associated with an AAA in the MVP cohort analysis of 9,693 AAA cases and 294,049 non-AAA controls (OR=1.03[95% CI:1.01-1.05, P=2.6×10⁻³) using analysis of individual level data with logistic regression. This was further supported through analysis using the UK Biobank (UKB) cohort with 97.6% European ancestry, with the association of PRS_{FMD} in AAAs being nominally significant with directionally consistent odds ratio (OR=1.065[95% CI:1.00-1.13], P=0.041) based on a smaller sample size of AAAs identified (998 AAA cases and 485,908 controls) as compared to MVP, using logistic regression of individual level data. Based upon simulations using similar effect sizes estimated from the data and the same sample size as in UKB, the association analyses of PRS_{FMD} and AAAs in the UKB cohort was quite underpowered, especially for a sex-stratified analysis (34.6% power for 876 AAA male cases versus 221,966 male controls) and therefore such was not performed. Sex-stratified analyses of the PRS_{FMD} association with AAAs in the MVP cohort was underpowered for women, with only 60 women with AAAs. An analysis of 9,633 men with AAAs and 272,758 controls, revealed a similar association as the analysis of both sexes combined (OR=1.03[95% CI:1.01-1.05, P=2.1×10⁻³). Next, the association of AAAs with PRS_{CarAD} (5 SNPs) and PRS_{SCAD} (7 SNPs) were examined, given that fathers of FMD probands with arterial dissections had an elevated RR_{AAA}. These analyses showed similar associations (OR=1.04[95% CI:1.01-1.06], P=8.4×10⁻⁴; and OR=1.03[95% CI:1.01-1.05], P=2.46×10⁻³ respectively) (Table 7, Table 9). Inspection of the 26 SNPs in the PRS_{FMD} in AAAs demonstrated associations with AAAs meeting P<0.05/26 at the chr9p21 and chr12q13.3 loci, and conversely of the 29 SNPs in the PRS_{AAA}, the same two loci met P<0.05/29 for association with FMD. A binomial test based on the finding of two SNPs associated with AAAs (P<0.05/26) among the 26 FMD-associated SNPs supported the significance of these shared associations (two-sided exact binomial test P-value=1.049×10⁻⁵, with estimated probability of reaching significance for both AAAs and FMD=0.077.

TABLE 6
26 FMD top ranked SNPs from the FMD GWAS cohort.
SNP RsID

rs9349379
rs6580732
rs4719277
rs72802873
rs59610103
rs2616437
rs7895641
rs6711554
rs7072877
rs1341809
rs73437338
rs17489499
rs6442124
rs944797
rs73684699
rs11075223
rs7625090
rs7898456
rs2294476
rs2664354
rs73055680
rs11172113
rs6763419
rs342402
rs13196590
rs12041871

TABLE 7
Summary of polygenic risk score (PRS) analyses. PRS analysis in FMD using known AAA SNPs based
on FMD GWAS samples, and PRS analysis in AAA using known FMD, CeAD, or SCAD SNPs based on Million
Veteran Program (MVP) AAA GWAS samples. PRS tests were constructed based upon logistic regression
of individual genotypes, adjusting for age, sex (if applicable) and the first five PCs, except
where indicated by “GTX,” the PRS was derivative of summary statistics using the GTX program.
Both logistic regression and GTX methods yielded similar PRS association results. The reported PRS
p-values were un-adjusted, and when meeting the Bonferroni-corrected significance threshold (0.05/10 = 0.005)
the result is shown in bold font. For replication purposes, the association of PRS_{FMD} and AAA trait
was tested in the UK Biobank (UKB) cohort. The replication p-value thresohold after multiple test
correction was 0.05/1 = 0.05. Despite of the smaller sample size and limited power, the replication
in UKB was positive (p-value = 0.04) and displayed a similar odds ratio as that in the MVP analysis.

| | | | | | | | | | |
|---|---|---|---|---|---|---|---|---|---|
| | | | Total | | | PRS | PRS | PRS | PRS P- |
| Phenotype | Cohort | PRS* | N | N cases | N controls | β | SE | OR[95% CI] | value |

PRS_{AAA} association with FMD

| | | | | | | | | | |
|---|---|---|---|---|---|---|---|---|---|
| FMD | UM | PRS_{AAA}^{†} | 4,011 | 573 | 3,438 | 0.40 | 0.11 | 1.50[1.20-1.87] | 3.07E−04 |
| FMD | UM | PRS_{AAA}-GTX | 7,777 | 584 | 7,193 | 0.43 | 0.11 | 1.53[1.24-1.90] | 9.04E−05 |

PRS_{FMD} association with AAA in the MVP

| | | | | | | | | | |
|---|---|---|---|---|---|---|---|---|---|
| AAA-all | MVP | PRS_{FMD} | 303,742 | 9,693 | 94,049 | 0.03 | 0.01 | 1.03[1.01-1.05] | 2.62E−03 |
| AAA-male | MVP | PRS_{FMD} | 282,391 | 9,633 | 272,758 | 0.03 | 0.01 | 1.03[1.01-1.05] | 2.11E−03 |
| AAA-female | MVP | PRS_{FMD} | 21,351 | 60 | 21,291 | −0.10 | 0.13 | 0.91[0.70-1.18] | 0.48 |

PRSS_{CAD} association with AAA in the MVP

| | | | | | | | | | |
|---|---|---|---|---|---|---|---|---|---|
| AAA-all | MVP | PRSS_{CAD} | 303,742 | 9,693 | 294,049 | 0.03 | 0.01 | 1.03[1.01-1.05] | 2.46E−03 |
| AAA-male | MVP | PRSS_{CAD} | 282,391 | 9,633 | 272,758 | 0.03 | 0.01 | 1.03[1.01-1.05] | 2.01E−03 |
| AAA-female | MVP | PRSS_{CAD} | 21,351 | 60 | 21,291 | −0.08 | 0.13 | 0.92[0.71-1.19] | 0.53 |

PRS_{CeAD} association with AAA in the MVP

| | | | | | | | | | |
|---|---|---|---|---|---|---|---|---|---|
| AAA-all | MVP | PRS_{CeAD} | 303,742 | 9,693 | 294,049 | 0.03 | 0.01 | 1.04[1.01-1.06] | 8.39E−04 |
| AAA-male | MVP | PRS_{CeAD} | 282,391 | 9,633 | 272,758 | 0.03 | 0.01 | 1.03[1.01-1.06] | 1.01E−03 |
| AAA-female | MVP | PRS_{CeAD} | 21,351 | 60 | 21,291 | 0.09 | 0.13 | 1.10[0.86-1.41] | 0.46 |

Replication analysis: PRS_{FMD} association with AAA in UKB

| | | | | | | | | | |
|---|---|---|---|---|---|---|---|---|---|
| AAA-all | UKB | PRS_{FMD} | 486,906 | 998 | 485,908 | 0.06 | 0.03 | 1.07[1.00-1.13] | 0.04 |

*Weighted PRS scores
^{†}Logistic regression PRS analysis of 573 FMD cases versus matched 3,438 MGI controls (1:6 case control ratio, after removing close relatives), adjusted for age, sex, PC1-PC5; (see eFigure 3 for PRS distribution plot).

TABLE 8
29 known AAA GWAS SNPs in MVP and their
individual SNP result of FMD GWAS.
SNP RsID

rs11591147
rs646776
rs12730935
rs4916254
rs7255
rs1399623
rs7628052
rs3176336
rs7742931
rs118039278
rs10808546
rs4007642
rs7025486
rs1412445
rs501630
rs1892971
rs964184
rs4936098
rs1581613
rs11172113
rs7994761
rs1271512
rs55958997
rs4401144
rs73015016
rs429358
rs8124182
rs73149487
rs2836411

TABLE 9
Top SCAD SNPs and CeAD SNPs based on published data.

CeAD SNPs rsID
rs12402265
rs6741522
rs6820391
rs9349379
rs11172113
SCAD SNPs rsID
rs12740679
rs11172113
rs9349379
rs28451064
rs78349783
rs78377252
rs11207415

### Discussion

Experiments conducted during development of embodiments herein documented an elevated risk of FMD among female relatives of FMD probands, particularly among relatives of probands exhibiting arterial dissections or aneurysms. Male and female relatives exhibited various forms of confirmed or suspected DAAD, supporting the tenet that DAADs are part of a shared systemic arteriopathy with variable manifestations of FMD. Notably, a new relationship between FMD and AAA risk was identified in our pedigrees, and a shared complex genetic architecture of FMD and AAAs was validated through PRS analyses among participants of large, genotyped cohorts.

Both FMD and AAAs are relatively common vascular diseases, with FMD affecting ˜3.3% of the population and AAAs affecting ˜4.9% of the population, yet there are important differences. FMD is approximately nine times more frequent in women, while an AAA is four-to 10-times more frequent in men. The mean age of FMD diagnosis in the United States is 53.3 years whereas approximately two-thirds of AAAs are recognized after age 75. Current screening guidelines for AAAs exist for all men age 65 years or older with any history of smoking, yet no such association has been established to support a screening guideline for FMD. While genetic associations for FMD are emerging, a complex genetic basis for AAAs has been better defined, and family history of an AAA is a recognized risk factor for AAAs.

FMD and AAAs appear to have shared as well as distinct vascular histopathologic features. The most notable similarity is extensive remodeling of the vascular extra-cellular matrix. Inflammation is not a feature of FMD, whereas AAAs exhibit marked inflammatory changes. A number of cytokine alterations have been described in a severe FMD cohort, but whether these promote arterial disease more broadly is unknown. Alterations in TGF-β signaling pathways in both AAAs and FMD may play a role in the extracellular matrix and SMC changes in both diseases, particularly in promoting vascular fibrosis, but a precise mechanistic role has not been defined.

The chromosome 6p24.1 PHACTR1 locus was the only genome-wide significant locus identified by the FMD GWAS, and showed no association with AAAs. However, among the 26 SNPs in the PRS_{FMD}), an association at the chromosome 12q13.3 LRP1 locus was found to have concordant genetic effects, with the same risk allele, across vascular outcomes including FMD, AAAs, SCAD and CarAD. Among AAA-associated loci in the 29-SNP PRS_{AAA}, the chromosome 9p21.3 locus was associated with FMD, although below genome-wide significance. In a murine model, LRP1 expression in arterial SMCs has been shown to impact cell migration and vascular wall integrity. While the FMD-, SCAD-, CarAD-, and AAA-associated allele is expected to raise LRP1 transcript expression, according to expression QTLs defined in human arterial tissues, and higher LRP1 expression is protective against arterial atherosclerosis, it is notable that actin polymerization and cellular contraction were abnormal in mice with smooth-muscle cell specific deletion of LRP1. Vascular SMC proliferation and apoptosis regulated at the 9p21 locus through expression regulation of CDKN2B has potential relevance for fibroproliferation in the arterial media and aneurysm formation.

FMD and AAAs have not previously been considered related diseases, given the low prevalence of an AAA among FMD patients. This supports a true sex dimorphism as men with an AAA have not been described to have FMD in the branch vessels of the aorta, and women with FMD, despite more frequent hypertension due to renovascular involvement, have not been reported to have any higher incidence of AAAs. The influence of reproductive hormones or sex chromosome differential gene expression may affect DAAD phenotypes, including FMD proband fathers with AAAs, and such requires further study. Given the previously reported shared associations between FMD, SCAD, and carotid artery dissection, these findings support the idea of shared genetic determinants playing a role in arterial dysplasia overall.

In some embodiments, the clinical implications of this study's findings relate to AAA screening, and indicate that screening could be extended to men with no history of smoking, if they have a first-degree family member with FMD. Tobacco smoking is estimated to increase the relative risk of having an AAA ˜1.87 per 10 cigarettes per day, which is comparable to the risk identified in the current study's FMD pedigree analysis. Screening men with a family history of FMD may counter the oversight of individuals having AAAs who are ineligible for screening under the current guidelines.

Experiments were conducted during development of embodiments herein to test the PRS-FMD in 342 individuals with SCAD. The PRS-FMD was associated with a diagnosis of SCAD (p=0.0107). Beta=0.28794551m stdev=0.112888391. This finding indicates that the PRS-FMD can discriminate risk of FMD in subjects with SCAD. In some embodiments, provided herein are methods of determining a subject's risk of FMD, wherein the subject suffers from SCAD, comprising calculating a polygenic risk score for the subject.

### REFERENCES

The following references, some of which are cited above by number, are herein incorporated by reference in their entireties.
1. Harrison E G, Jr. and McCormack L J. Pathologic classification of renal arterial disease in renovascular hypertension. Mayo Clin Proc. 1971:46:161-7.
2. Olin J W, Froehlich J, Gu X, Bacharach J M, Eagle K, Gray B H, Jaff M R, Kim E S, Mace P. Matsumoto A H, et al. The United States Registry for Fibromuscular Dysplasia: results in the first 447 patients. Circulation. 2012; 125:3182-90.
3. Olin J W, Gornik H L, Bacharach J M, Biller J. Fine L J, Gray B H, Gray W A, Gupta R, Hamburg N M, Katzen B T, et al. Fibromuscular dysplasia: state of the science and critical unanswered questions: a scientific statement from the American Heart Association. Circulation. 2014; 129:1048-78.
4. Sottiurai V S, Fry W J and Stanley J C. Ultrastructure of medial smooth muscle and myofibroblasts in human arterial dysplasia. Arch Surg. 1978; 113-1280-8.
5. Stanley J C, Gewertz B L, Bove E L, Sottiurai V and Fry W J. Arterial fibrodysplasia. Histopathologic character and current etiologic concepts. Arch Surg. 1975; 110:561-6.
6. Gomik H L, Persu A, Adlam D, Aparicio L S, Azizi M, Boulanger M, Bruno R M, de Leeuw P, Fendrikova-Mahlay N, Froehlich J, et al. First International Consensus on the diagnosis and management of fibromuscular dysplasia. Vasc Med. 2019; 24:164-189.
7. Savard S, Steichen O, Azarine A, Azizi M, Jeunemaitre X and Plouin P F Association between 2 angiographic subtypes of renal artery fibromuscular dysplasia and clinical characteristics. Circulation. 2012; 126:3062-9.
8. Olin J W and Pierce M. Contemporary management of fibromuscular dysplasia. Curr Opin Cardiol. 2008; 23:527-36.
9. Norman P E and Powell J T. Site specificity of aneurysmal disease. Circulation. 2010; 121:560-8.
10. Rushton A R. The genetics of fibromuscular dysplasia. Arch Intern Med. 1980; 140:233-6.
11. Gladstien K, Rushton A R and Kidd K K. Penetrance estimates and recurrence risks for fibromuscular dysplasia. Clin Genet. 1980; 17:115-6.
12. Debette S, Kamatani Y, Metso T M, Kloss M, Chauhan G, Engelter S T, Pezzini A, Thijs V, Markus H S, Dichgans M, et al. Common variation in PHACTR1 is associated with susceptibility to cervical artery dissection. Nat Genet. 2015; 47:78-83.
13. Kiando S R, Tucker N R, Castro-Vega L J, Katz A, D'Escamard V, Treard C, Fraher D, Albuisson J, Kadian-Dodov D, Ye Z, et al. PHACTR1 Is a Genetic Susceptibility Locus for Fibromuscular Dysplasia Supporting Its Complex Genetic Pattern of Inheritance. PLoS Genet. 2016; 12:e1006367.
14. Saw J, Yang M L, Trinder M, Tcheandjieu C, Xu C, Starovoytov A, Birt I, Mathis M R, Hunker K L, Schmidt E M, et al. Chromosome 1q21.2 and additional loci influence risk of spontaneous coronary artery dissection and myocardial infarction. Nat Commun. 2020; 11:4432.
15. Ganesh S K, Morissette R. Xu Z, Schoenhoff F, Griswold B F, Yang J, Tong L, Yang M L, Hunker K, Sloper L, et al. Clinical and biochemical profiles suggest fibromuscular dysplasia is a systemic disease with altered TGF-beta expression and connective tissue features. FASEB J. 2014:28:3313-24.
16. Shivapour D M, Erwin P and Kim E. Epidemiology of fibromuscular dysplasia: A review of the literature. Vasc Med. 2016; 21:376-81.
17. Davis F M, Daugherty A and Lu H S. Updates of Recent Aortic Aneurysm Research. Arterioscler Thromb Vasc Biol. 2019; 39:e83-e90.
18. Forsdahl S H, Singh K, Solberg S and Jacobsen B K. Risk factors for abdominal aortic aneurysms: a 7-year prospective study: the Tromso Study, 1994-2001. Circulation. 2009; 119:2202-8.
19. Howard D P, Banerjee A, Fairhead J F, Handa A, Silver L E, Rothwell P M and Oxford Vascular S. Age-specific incidence, risk factors and outcome of acute abdominal aortic aneurysms in a defined population. Br J Surg 2015; 102:907-15.
20. Force USPST, Owens D K, Davidson K W. Krist A H, Barry M J, Cabana M, Caughey A B, Doubeni C A, Epling J W, Jr., Kubik M, et al. Screening for Abdominal Aortic Aneurysm: US Preventive Services Task Force Recommendation Statement. JAMA. 2019; 322:2211-2218.
21. O'Donnell T F X and Schermerhorn M L. Abdominal aortic aneurysm screening guidelines: United States Preventative Services Task Force and Society for Vascular Surgery. J Vasc Surg. 2020; 71:1457-1458.
22. Guirguis-Blake J M, Beil T L, Senger C A and Coppola E L. Primary Care Screening for Abdominal Aortic Aneurysm: Updated Evidence Report and Systematic Review for the US Preventive Services Task Force. JAMA. 2019; 322:2219-2238.
23. Chaikof E L, Dalman R L, Eskandari M K, Jackson B M, Lee W A, Mansour M A, Mastracci T M, Mell M, Murad M H, Nguyen L L, et al. The Society for Vascular Surgery practice guidelines on the care of patients with an abdominal aortic aneurysm. J Vase Surg. 2018; 67:2-77 e2.
24. Bown M J, Jones G T, Harrison S C, Wright B J, Bumpstead S, Baas A F, Gretarsdottir S, Badger S A, Bradley D T, Burnand K, et al. Abdominal aortic aneurysm is associated with a variant in low-density lipoprotein receptor-related protein 1. Am J Hum Genet. 2011; 89:619-27.
25. Bradley D T, Hughes A E, Badger S A, Jones G T, Harrison S C, Wright B J, Bumpstead S, Baas A F, Gretarsdottir S, Burnand K, et al. A variant in LDLR is associated with abdominal aortic aneurysm. Circ Cardiovase Genet. 2013; 6:498-504.
26. Elmore J R, Obmann M A, Kuivaniemi H, Tromp G, Gerhard G S, Franklin D P, Boddy A M and Carey D J. Identification of a genetic variant associated with abdominal aortic aneurysms on chromosome 3p12.3 by genome wide association. J Vasc Surg. 2009; 49.1525-31
27. Gretarsdottir S, Baas A F, Thorleifsson G, Holm H, den Heijer M, de Vries J P, Kranendonk S E, Zeebregts C J, van Sterkenburg S M, Geelkerken R H, et al. Genome-wide association study identifies a sequence variant within the DAB2IP gene conferring susceptibility to abdominal aortic aneurysm. Nat Genet. 2010:42:692-7.
28. Jones G T, Tromp G. Kuivaniemi H, Gretarsdottir S, Baas A F, Giusti B, Strauss E, Van't Hof F N, Webb T R, Erdman R, et al. Meta-Analysis of Genome-Wide Association Studies for Abdominal Aortic Aneurysm Identifies Four New Disease-Specific Risk Loci. Circ Res. 2017; 120:341-353.
29. Klarin D, Verma S S, Judy R, Dikilitas O, Wolford B N, Paranjpe I, Levin M G, Pan C, Tcheandjieu C, Spin J M, et al. Genetic Architecture of Abdominal Aortic Aneurysm in the Million Veteran Program. Circulation. 2020; 142:1633-1646.
30. Li J, Pan C, Zhang S, Spin J M, Deng A, Leung L L K, Dalman R L, Tsao P S and Snyder M. Decoding the Genomics of Abdominal Aortic Aneurysm. Cell. 2018; 174:1361-1372 e10.
31. van't Hof F N, Ruigrok Y M, Lee C H, Ripke S, Anderson G, de Andrade M, Baas A F, Blankensteijn J D, Bottinger E P, Bown M J, et al. Shared Genetic Risk Factors of Intracranial, Abdominal, and Thoracic Aneurysms. J Am Heart Assoc 2016; 5.
32. Chang Z, Zhao G, Zhao Y, Lu H, Xiong W, Liang W, Sun J, Wang H, Zhu T, Rom O, et al. BAF60a Deficiency in Vascular Smooth Muscle Cells Prevents Abdominal Aortic Aneurysm by Reducing Inflammation and Extracellular Matrix Degradation. Arterioscler Thromb Vasc Biol. 2020; 40:2494-2507.
33. Doyle A J, Redmond E M, Gillespie D L, Knight P A, Cullen J P, Cahill P A and Morrow D J. Differential expression of Hedgehog/Notch and transforming growth factor-beta in human abdominal aortic aneurysms. J Vasc Surg. 2015; 62:464-70.
34. Wang Y, Ait-Oufella H, Herbin O, Bonnin P, Ramkhelawon B, Taleb S, Huang J, Offenstadt G, Combadiere C, Renia L, et al. TGF-beta activity protects against inflammatory aortic aneurysm progression and complications in angiotensin II-infused mice. J Clin Invest. 2010; 120:422-32.
35. Davis F M, Rateri D L and Daugherty A. Abdominal aortic aneurysm: novel mechanisms and therapies. Curr Opin Cardiol. 2015; 30:566-73.
36. Boucher P, Gotthardt M, Li W P, Anderson R G and Herz J. LRP: role in vascular wall integrity and protection from atherosclerosis. Science. 2003; 300.329-32.
37. Au D T, Ying Z, Hernandez-Ochoa E O, Fondrie W E, Hampton B, Migliorini M, Galisteo R, Schneider M F, Daugherty A, Rateri D L, et al. LRP1 (Low-Density Lipoprotein Receptor-Related Protein 1) Regulates Smooth Muscle Contractility by Modulating Ca(2+) Signaling and Expression of Cytoskeleton-Related Proteins. Arterioscler Thromb Vase Biol. 2018; 38:2651-2664.
38. Miller C L, Pjanic M, Wang T, Nguyen T, Cohain A, Lee J D, Perisic L, Hedin U, Kundu R K, Majmudar D, et al. Integrative functional genomics identifies regulatory mechanisms at coronary artery disease loci. Nat Commun. 2016; 7:12092.
39. Leeper N J, Raiesdana A, Kojima Y, Kundu R K, Cheng H, Maegdefessel L, Toh R, Ahn G O, Ali Z A, Anderson D R, et al. Loss of CDKN2B promotes p53-dependent smooth muscle cell apoptosis and aneurysm formation. Arterioscler Thromb Vasc Biol. 2013:33:e1-e10.
40. Norris C M, Yip C Y Y, Nerenberg K A, Clavel M A, Pacheco C, Foulds H J A, Hardy M, Gonsalves C A, Jaffer S, Parry M, et al. State of the Science in Women's Cardiovascular Disease: A Canadian Perspective on the Influence of Sex and Gender. J Am Heart Assoc. 2020; 9:e015634.
41. Pacheco Claudio C, Quesada O, Pepine C J and Noel Bairey Merz C. Why names matter for women: MINOCA/INOCA (myocardial infarction/ischemia and no obstructive coronary artery disease) Clin Cardiol. 2018; 41:185-193.
42. Hayes S N, Kim E S H, Saw J, Adlam D, Arslanian-Engoren C, Economy K E, Ganesh S K, Gulati R, Lindsay M E, Mieres J H, et al. Spontaneous Coronary Artery Dissection: Current State of the Science: A Scientific Statement From the American Heart Association. Circulation. 2018; 137:e523-e557.
43. Kim E S H, Olin J W, Froehlich J B, Gu X, Bacharach J M, Gray B H, Jaff M R, Katzen B T, Kline-Rogers E, Mace P D, et al. Clinical manifestations of fibromuscular dysplasia vary by patient sex: a report of the United States registry for fibromuscular dysplasia. J Am Coll Cardiol 2013; 62:2026-2028.
44. Saw J, Aymong E, Sedlak T, Buller C E, Starovoytov A, Ricci D, Robinson S. Vuurmans T, Gao M, Humphries K, et al. Spontaneous coronary artery dissection: association with predisposing arteriopathies and precipitating stressors and cardiovascular outcomes. Circ Cardiovasc Interv. 2014; 7:645-55.
45. Kadian-Dodov D, Gornik H L, Gu X, Froehlich J, Bacharach J M, Chi Y W, Gray B H, Jaff M R, Kim E S, Mace P, et al. Dissection and Aneurysm in Patients With Fibromuscular Dysplasia: Findings From the U.S. Registry for FMD. J Am Coll Cardiol. 2016:68:176-85.
46. Henriques T, Zhang X, Yiannikouris F B, Daugherty A and Cassis L A. Androgen increases AT1a receptor expression in abdominal aortas to promote angiotensin II-induced AAAs in apolipoprotein E-deficient mice. Arterioscler Thromb Vasc Biol. 2008; 28:1251-6.
47 Oliva M, Munoz-Aguirre M, Kim-Hellmuth S, Wucher V, Gewirtz A D H, Cotter D J, Parsana P, Kasela S, Balliu B, Vinuela A, et al. The impact of sex on gene expression across human tissues. Science. 2020; 369.
48. Aune D, Schlesinger S, Norat T and Riboli E. Tobacco smoking and the risk of abdominal aortic aneurysm: a systematic review and meta-analysis of prospective studies. Sci Rep. 2018; 8:14786.
49. Kent K C, Zwolak R M, Egorova N N, Riles T S, Manganaro A, Moskowitz A J, Gelijns A C and Greco G. Analysis of risk factors for abdominal aortic aneurysm in a cohort of more than 3 million individuals. J Vasc Surg. 2010; 52:539-48
50. Rana M N and Al-Kindi S G. Prevalence and manifestations of diagnosed fibromuscular dysplasia by sex and race. Analysis of >4500 FMD cases in the United States. Heart Lung. 2020.
51. Boll A P, Verbeek A L, van de Lisdonk E H and van der Vliet J A. High prevalence of abdominal aortic aneurysm in a primary care screening programme. Br J Surg. 1998:85:1090-4.
52. Lederle F A, Johnson G R, Wilson S E, Chute E P, Littooy F N, Bandyk D, Krupski W C, Barone G W, Acher C W and Ballard D J. Prevalence and associations of abdominal aortic aneurysm detected through screening. Aneurysm Detection and Management (ADAM) Veterans Affairs Cooperative Study Group. Ann Intern Med. 1997; 126:441-9.
53. Scott R A, Ashton H A and Kay D N. Abdominal aortic aneurysm in 4237 screened patients: prevalence, development and management over 6 years. Br J Surg. 1991; 78:1122-5.
54. Fritsche L G, Gruber S B, Wu Z, Schmidt E M, Zawistowski M, Moser S E, Blanc V M, Brummett C M, Kheterpal S, Abecasis G R, et al Association of Polygenic Risk Scores for Multiple Cancers in a Phenome-wide Study: Results from The Michigan Genomics Initiative. Am J Hum Genet. 2018; 102:1048-1061.
55. Gaziano J M, Concato J, Brophy M, Fiore L, Pyarajan S, Breeling J, Whitbourne S, Deen J, Shannon C, Humphries D, et al. Million Veteran Program: A mega-biobank to study genetic influences on health and disease. J Clin Epidemiol. 2016; 70:214-23.
56. Fang H, Hui Q, Lynch J, Honerlaw J, Assimes T L, Huang J, Vujkovic M, Damrauer S M, Pyarajan S, Gaziano J M, et al. Harmonizing Genetic Ancestry and Self-identified Race/Ethnicity in Genome-wide Association Studies. Am J Hum Genet. 2019; 105:763-772.
57. Hunter-Zinck H, Shi Y, Li M, Gorman B R, Ji S-G, Sun N, Webster I, Liem A, Hsieh P, Devineni P, et al. Measuring genetic variation in the multi-ethnic Million Veteran Program (MVP). bioRxiv. 2020:2020.01.06.896613.
58. Tcheandjieu C, Xiao K, Tejeda H, Lynch J A, Ruotsalainen S, Bellomo T, Palnati M, Judy R, Klarin D, Kember R, et al. High heritability of ascending aortic diameter and multi-ethnic prediction of thoracic aortic disease. medRxiv. 2020: 2020.05.29.20102335.
59. Zhou W, Nielsen J B, Fritsche L G, Dey R, Gabrielsen M E, Wolford B N, LeFaive J, VandeHaar P, Gagliano S A, Gifford A, et al. Efficiently controlling for case-control imbalance and sample relatedness in large-scale genetic association studies. Nat Genet. 2018; 50:1335-1341.
60. Li J Z. Absher D M, Tang H, Southwick A M, Casto A M, Ramachandran S, Cann H M, Barsh G S, Feldman M, Cavalli-Sforza L L, et al. Worldwide human relationships inferred from genome-wide patterns of variation. Science. 2008; 319:1100-4.
61. Taliun D, Chothani S P, Schonherr S. Forer L, Bochnke M, Abecasis G R and Wang C. LASER server: ancestry tracing with genotypes or sequence reads. Bioinformatics 2017; 33:2056-2058.
62. Wang C, Zhan X, Liang L, Abecasis G R and Lin X. Improved ancestry estimation for both genotyping and sequencing data using projection procrustes analysis and genotype imputation. Am J Hum Genet. 2015; 96:926-37.
63. Wang C, Zhan X, Bragg-Gresham J, Kang H M, Stambolian D, Chew E Y, Branham K E, Heckenlively J, Study F, Fulton R, et al. Ancestry estimation and control of population stratification for sequence-based association studies. Nat Genet. 2014; 46:409-15.
64. Dastani Z, Hivert M F, Timpson N, Perry J R, Yuan X, Scott R A, Henneman P. Heid I M, Kizer J R, Lyytikainen L P, et al. Novel loci for adiponectin levels and their influence on type 2 diabetes and metabolic traits: a multi-ethnic meta-analysis of 45,891 individuals. PLoS Genet. 2012; 8:e1002607.
65. International Consortium for Blood Pressure Genome-Wide Association S, Ehret G B, Munroe P B, Rice K M, Bochud M, Johnson A D, Chasman D I, Smith A V, Tobin M D, Verwoert G C, et al. Genetic variants in novel pathways influence blood pressure and cardiovascular disease risk. Nature. 2011; 478:103-9.
66. Johnson T. Genetic risk score calculation from summary statistics. May 2, 2019.

1. A method of assessing the risk of fibromuscular dysplasia (FMD) comprising:
(a) testing a sample from the subject for biomarkers of FMD; and
(b) assessing the subject's risk of FMD.

2. The method of claim 1, wherein the biomarkers of FMD are selected from rs9349379 rs6580732, rs4719277, rs72802873, rs59610103, rs2616437, rs7895641, rs6711554, rs7072877, rs1341809, rs73437338, rs17489499, rs6442124, rs944797, rs73684699, rs11075223, rs7625090, rs7898456, rs2294476, rs2664354, rs73055680, rs11172113, rs6763419, rs342402, rs13196590, and rs12041871.

3. The method of claim 2, wherein any combination of two or more of rs9349379 rs6580732, rs4719277, rs72802873, rs59610103, rs2616437, rs7895641, rs6711554, rs7072877, rs1341809, rs73437338, rs17489499, rs6442124, rs944797, rs73684699, rs11075223, rs7625090, rs7898456, rs2294476, rs2664354, rs73055680, rs11172113, rs6763419, rs342402, rs13196590, and rs12041871 are analyzed.

4. The method of claim 1, wherein assessing the subject's risk of FMD comprises:
(i) calculating a risk score based on the biomarkers for FMD; and
(ii) comparing the risk score to a threshold to determine the subject's risk for FMD.

5. The method of claim 4, wherein the presence of any biomarker is weighted according to its odds ratio in order to calculate the risk score.

6. The method of claim 1, wherein the subject suffers from spontaneous coronary artery dissection (SCAD).

7. The method of claim 1, wherein the subject is a human female.

8. The method of claim 1, wherein the subject as a first or second degree relative that suffers from or has suffered from fibromuscular dysplasia (FMD) or AAA.

9. A method of assessing the risk of abdominal aortic aneurysm (AAA) comprising:
(a) testing a sample from the subject for biomarkers of AAA; and
(b) assessing the subject's risk of AAA.

10. The method of claim 9, wherein the biomarkers of AAA are selected from rs11591147 rs646776, rs12730935, rs4916254, rs7255, rs1399623, rs7628052, rs3176336, rs7742931, rs118039278, rs10808546, rs4007642, rs7025486, rs1412445, rs501630, rs1892971, rs964184, rs4936098, rs1581613, rs11172113, rs7994761, rs1271512, rs55958997, rs4401144, rs73015016, rs429358, rs8124182, rs73149487, and rs2836411.

11. The method of claim 10, wherein any combination of two or more of rs11591147 rs646776, rs12730935, rs4916254, rs7255, rs1399623, rs7628052, rs3176336, rs7742931, rs118039278, rs10808546, rs4007642, rs7025486, rs1412445, rs501630, rs1892971, rs964184, rs4936098, rs1581613, rs11172113, rs7994761, rs1271512, rs55958997, rs4401144, rs73015016, rs429358, rs8124182, rs73149487, and rs2836411 are analyzed.

12. The method of claim 9, wherein assessing the subject's risk of AAA comprises:
(i) calculating a risk score based on the biomarkers for AAA; and
(ii) comparing the risk score to a threshold to determine the subject's risk for AAA.

13. The method of claim 12, wherein the presence of any biomarker is weighted according to its odds ratio in order to calculate the risk score.

14. The method of claim 9, wherein the subject is a human male.

15. The method of claim 14, wherein the subject as a first or second degree relative that suffers from or has suffered from fibromuscular dysplasia (FMD) or AAA.

16. A method of preventing fibromuscular dysplasia (FMD) comprising:
(1) assessing the risk of FMD by the method of one of claims 1-8; and
(2) administering a prophylactic and/or symptom management to reduce the subject's risk for developing FMD.

17. The method of claim 16, wherein the prophylactic and/or symptom management comprises one or more of:
(i) administering antiplatelet therapy;
(ii) cessation of tobacco use;
(iii) balloon angioplasty;
(iv) percutaneous transluminal angioplasty; and
(v) arteriogram.

18. A method of preventing abdominal aortic aneurysm (AAA) comprising:
(1) assessing the risk of AAA by the method of one of claims 9-15; and
(2) administering a prophylactic and/or symptom management regime to reduce the subject's risk for developing AAA.

19. The method of claim 18, wherein the prophylactic and/or symptom management comprises one or more of:
(i) avoidance of exogenous hormone exposure;
(ii) smoking cessation;
(iii) imaging surveillance;
(iv) avoiding activities that increase arterial strain or spikes in blood pressure;
(v) monitoring blood pressure;
(vi) administering a beta blocker;
(vii) administering an angiotensin-converting enzyme inhibitor (ACEI);
(viii) counseling of family members to seek evaluation for vascular symptoms; and
(ix) administering an angiotensin II receptor blocker (ARB).

20. A method comprising:
(a) identifying a male subject as a first or second degree relative of a female individual that suffers from or has suffered from FMD; and
(b) assessing the risk of AAA for the subject by the method of one of claims 9-15.

21. A method comprising:
(a) identifying a female subject as a first or second degree relative of an individual that suffers from or has suffered from AAA; and
(b) assessing the risk of FMD for the subject by the method of one of claims 8-11.
